(19) United States
(12) Patent Application Publication
(10) Pub. No.: US 2025/0318743 A1
(43) Pub. Date: Oct. 16, 2025

(54) METHOD FOR DETERMINING A FIRST MOTION PHASE, METHOD FOR CORRECTING A TRIGGERING, MAGNETIC RESONANCE DEVICE, COMPUTER PROGRAM PRODUCT AND A COMPUTER-READABLE STORAGE MEDIUM

(71) Applicants: Siemens Healthineers AG, Forchheim (DE); The Ohio State University, Columbus, OH (US)

(72) Inventors: Peter Speier, Erlangen (DE); Yue Pan, Columbus, OH (US); Orlando P. Simonetti, Columbus, OH (US); Rizwan Ahmad, Columbus, OH (US)

(21) Appl. No.: 18/636,895

(22) Filed: Apr. 16, 2024

(51) Int. Cl. A61B 5/055; A61B 5/00; G16H 50/20

(52) U.S. Cl. CPC A61B 5/055; A61B 5/0044; A61B 5/7267; G16H 50/20; A61B 2576/023

(57) ABSTRACT

Systems and methods for determining a first motion pattern of a subsequent first motion period, including the following steps performed by a control unit of a magnetic resonance device for each current first motion period: extracting a pilot tone raw signal from the raw data signal; extracting at least one first motion component from the pilot tone raw signal; determining a respective first motion signal that is a first derivative of the at least one first motion component; determining a determined activity indicator value of the current first motion period, that is a maximum amplitude value of the respective first motion signal of the current first motion period; determining a predicted activity indicator value of the subsequent first motion period based on the determined activity indicator value of the current first motion period.

### FIELD

Embodiments relate to a method for determining a first motion pattern, a method for adapting a cardiac triggering procedure, a magnetic resonance device, a computer program product, and a computer-readable storage medium.

### BACKGROUND

For the cardiac triggering procedure it is necessary to observe a cardiac cycle of a patient. A common method to observe the cardiac cycle is electrocardiogram preparation (ECG). Another method is a cardiac triggering procedure based on a pilot tone (CPT), that is desirable because it eliminates the need for electrocardiogram preparation, is flexible because it is based on a continuous representation of cardiac motion and is insensitive to gradient interference.

The cardiac triggering procedure based on pilot tone (CPT) is described, for example, in US 2023/0079852 A1.

One drawback of the cardiac triggering procedure based on pilot tone is that an extraction algorithm of the cardiac signal, CPT signal, from a raw pilot tone data must be trained and that this training is valid only for similar situations. The situation is related to a current breathing of the patient. Current breathing may be described by a current breathing pattern related to a current phase of breathing. The CPT signal is used as a trigger signal. Normal training under free breathing is robust under this condition and during breath holds in exhale position as free breathing exhalation has a similar geometry. However, inhalation for breath holds in inhale position is very variable and often much deeper than during free breathing and thus not well covered by the training. During these non-trained situations, a signal extraction may be suboptimal, leading to a reduced amplitude of the trigger signal. This amplitude reduction may cause problems with current trigger algorithms. A robust threshold-based trigger detection algorithm triggers at a fixed fraction (0.4) of a trained amplitude. The reduced amplitude of the trigger signal may result therefore in a delayed trigger detection or, if the amplitude falls below the threshold, to missed triggers.

The solution so far is a strong user recommendation to use exhale-breath holds instead of inhale-breath holds when the cardiac triggering procedure based on pilot tone is used.

### BRIEF SUMMARY AND DESCRIPTION

The scope of the present disclosure is defined solely by the claims and is not affected to any degree by the statements within this summary. The present embodiments may obviate one or more of the drawbacks or limitations in the related art. Independent of the grammatical term usage, individuals with male, female or other gender identities are included within the term.

Embodiments provide a solution that minimizes the impairment of trigger detection by breathing.

Embodiments provide a real-time correction of amplitude variations of the trigger signal to stabilize a trigger detection during breath holds in inhale position without compromising the trigger detection during free breathing and breath holds in exhale position.

A first aspect relates to a method for determining a first motion pattern of a subsequent first motion period of a current first motion period of a cyclic first motion of an object by a magnetic resonance device. In other words, an object may perform the cyclic first motion. The cyclic first motion may be related to a respiration of the object. The cyclic first motion may include a sequence of first motion periods. The respective first motion periods may be attributed to the first motion patterns. The first motion patterns may be related to specific patterns of breathing. The first motion patterns may include a free breathing pattern a deep breathing pattern and a breath hold pattern. The method is configured to determine the first motion patterns of the respective first motion periods.

The method includes the following steps performed by a control unit of the magnetic resonance device for each current first motion period of the cyclic first motion. In other words, the steps described below are performed by the control unit for each of the first motion periods of the cyclic first motion. The control unit is a control unit of the magnetic resonance device. The magnetic resonance device may be configured for magnetic resonance imaging of the object.

In each current first motion period the control unit receives a raw data signal acquired by a magnetic resonance receiver coil assembly of the magnetic resonance device. The raw data signal may include a pilot Tone raw signal and a magnetic resonance imaging signal. To analyse the first motion of the object, the pilot tone raw signal is used. Therefore, the method includes a step of extracting the pilot tone raw signal from the raw signal. The extraction may be performed according to a predefined pilot tone extraction method known from the state of the art.

The pilot tone raw signal may include different components attributed to respective motions of the object. To analyse the first motion of the object at least one first motion component of the pilot tone raw signal is extracted from the pilot tone raw signal. It may be possible that a dominant motion component related to the first motion is selected as the at least one first motion component. It may also be possible that several first motion components of the pilot tone raw signal are extracted from the pilot tone raw signal.

To analyse the first motion, a first motion signal is used. The first motion signal may be a cleaned or filtered first motion component of the of the pilot tone raw signal. The first motion signal may be evaluated by a high pass filter, or baseline and drift removal methods like a sliding average subtraction method. The first motion signal may be a first derivative of the at least one first motion component, or alternatively, a cleaned-up filtered first motion component of the of the pilot tone raw signal itself. The clean-up may include a high pass filtering of the filtered first motion component of the of the pilot tone raw signal.

The method includes a determination of a determined activity indicator value of the current first motion period, prior to the subsequent first motion period. The determined activity indicator value depends on the first motion signal of the current first motion period. The determined activity indicator value of the current first motion period may depend on a maximum amplitude, a median amplitude, an average amplitude, or a standard deviation of the first motion signal of the current first motion period. In other words, the first motion signal of the current first motion period is analysed to determine the determined activity indicator value based on the first motion signal of the current first motion period.

The method includes a determination of a predicted activity indicator value of the subsequent first motion period based on the determined activity indicator value of the current first motion period. In other words, the determined activity indicator value of the current first motion period is used to predict the predicted activity indicator value of the subsequent first motion period.

In a next step, the first motion pattern of the subsequent first motion period is identified as a function of the predicted activity indicator value of the subsequent first motion period. In other words, the predicted activity indicator value of the subsequent first motion period is used by the control unit to determine the first motion pattern of the subsequent first motion pattern. As an example, the first motion patterns may be attributed to specific first motion activities of the respective first motion pattern indicated by the activity indicator value of the respective pattern. As it may be necessary to know the first motion pattern at a beginning of the respective first motion pattern, the determination of the first motion pattern of the respective first motion period based on the determined activity indicator value of the respective first motion period may be too late. Therefore, the predicted activity indicator value of the subsequent first motion period is evaluated as a base to determine the first motion pattern of the respective first motion period. This is possible, because predefined relations between the indicator values of following first motion periods may be assumed.

According to a further embodiment, the respective first motion signal is a first derivative of the at least one first motion component; and wherein the determined activity indicator value of the current first motion period is a maximum amplitude value of the respective first motion signal of the current first motion period. In other words, the first motion signal is generated by the control unit by generating a first derivative of the at least one first motion component. In other words, the first motion signal is the first derivative of the at least one first motion component against a time. As the at least one first motion component changes during motion activities, the first derivative is a suitable parameter to observe the cyclic first motion. The determined activity indicator value of the current first motion period is a maximum amplitude value of the first motion signal of the current first motion period. In other words, the amplitude of the first motion signal of the current first motion period is analysed to find the maximum amplitude value of the amplitude of the first motion signal of the current first motion period. The maximum amplitude value of the amplitude of the first motion signal of the current first motion period is defined as the determined activity indicator value of the current first motion period.

According to a further embodiment, the respective first motion signal is a cleaned-up version of the at least one first motion component. The cleaned-up version may be the at least one first motion component after a use of a high pass filter on the at least one first motion component. The determined activity indicator value of the current first motion period is a mean or maximum amplitude value of the respective first motion signal of the current first motion period. In other words, the determination of the determined activity indicator value includes an investigation of the mean or maximum amplitude value of the respective first motion signal of the current first motion period. The mean or maximum amplitude value of the respective first motion signal is determined as the determined activity indicator value of the current first motion period.

According to a further embodiment, the method includes a training procedure, including the following steps performed by the control unit of the magnetic resonance device. The training procedure includes a step of recording the first motion signal during a training period associated with a predefined first motion pattern. The training procedure includes a step of evaluating at least one identification characteristic for identification of the predefined first motion pattern based on the first motion signal recorded during the training period. The at least one identification characteristic includes a length of a first motion period of the predefined first motion pattern and/or an average activity indicator value of the predefined first motion pattern and/or a standard deviation of the activity indicator value of the predefined first motion pattern and/or a direction of the respective first motion signal of the predefined first motion pattern.

According to a further embodiment, the method includes a step of identifying the first motion pattern of the subsequent first motion period as a function of the at least one identification characteristic.

According to a further embodiment, the method includes a comparison of the predicted activity indicator value of the subsequent first motion period to a predefined threshold value of the subsequent first motion period. The first motion pattern of the subsequent first motion period is identified depending on a result of the comparison. In other words, the identification of the first motion pattern of the subsequent first motion period is based on the result of the comparison of the predicted activity indicator value of the subsequent first motion period to the predefined threshold value of the subsequent first motion period. The predefined threshold value of the comparison may be preset or may be evaluated for the respective first motion period. The threshold could be learned during the training procedure with known respiratory pattern in a free breathing training. The result of the comparison may include that the predicted activity indicator value of the subsequent first motion period is above, below, or equal to the threshold value of the subsequent first motion period. The result may also include a distance of the predicted activity indicator value of the subsequent first motion period to the predefined threshold value of the subsequent first motion period. Depending on the result of the comparison, the respective first motion pattern of the subsequent first motion period may be identified. It may be possible that a respective one of the first motion patterns is assigned to the respective first motion period if the predicted activity indicator value is above the threshold value and that a respective one of the first motion patterns is assigned to the respective first motion period if the predicted activity indicator value is below the threshold value.

According to further embodiment, the threshold value of the subsequent first motion period is determined by the control unit based on determined activity indicator values of selected first motion periods of the first motion periods. In other words, the threshold value of the subsequent first motion period depends on the determined activity indicator values that were determined for the selected first motion periods. It may be possible that the threshold value is an average value of the determined indicator values of the selected first motion periods inside a moving window.

According to a further embodiment, the method includes a selection of the selected first periods from the first motion periods depending on the first motion pattern of the respective first motion period. In other words, the preceding first motion periods may be identified as respective first motion patterns. It may be possible that previous first motion periods that are identified as specific first motion patterns are defined as selected first motion periods. The determined activity indicator values of the selected first motion periods may be used to calculate the threshold value. The determined activity indicator values of previous first motion periods not identified as the selected periods may be ignored in the calculation of the threshold value.

According to a further embodiment, the first motion pattern of the subsequent first motion period is identified as a function of a length of the current first motion period. In other words, the control unit identifies the length of the current first motion period. Respective ones of the first motion patterns may be attributed to specific lengths of the first motion periods.

According to a further embodiment, the first motion pattern of the subsequent first motion period is identified as a function of the first motion pattern of the current first motion period. In other words, the first motion pattern of the subsequent pattern is identified as a function of the first motion pattern that is identified for the current first motion period. It may be possible that the first motion pattern of the current motion period is used along the predicted indicator value to identify the first motion pattern of the subsequent first motion period.

According to a further embodiment, the method includes an extraction of at least two of the first motion components from the pilot tone raw signal. In other words, a further first motion component may be extracted by the control unit. The control unit may determine a further respective first motion signal that may be a first derivative of the further first motion component, or alternatively, a cleaned-up filtered further first motion component of the of the pilot tone raw signal itself. The control unit may determine a further determined activity indicator value of the further current first motion period of the further first motion component. The further determined activity indicator value of the further current first motion period may depend on a maximum amplitude, a median amplitude, an average amplitude, or a standard deviation of the further first motion signal of the current first motion period. The control unit may determine a further predicted activity indicator value of the further subsequent first motion period based on the further determined activity indicator value of the further current first motion period. The control unit may identify the first motion phase of the further subsequent first motion period as a function of the further predicted activity indicator value of the further subsequent first motion period. The further first motion periods may be the same as the first motion periods. It may be possible that the first motion phase of the subsequent first motion period is identified as a function of the predicted activity indicator value and the further predicted activity indicator value. The advantage of this embodiment is that the first motion phase may be identified based on at least two inputs, reducing the number of misidentifications.

According to a further embodiment, the at least two of the first motion components are phase-shifted to each other. In other words, there is a phase shift between the first motion periods of the at least two first motion components. At the end of each of the first motion periods, the predicted activity indicator value of the subsequent first motion period may be determined. As the phases are shifted, a frequency of determination of the predicted activity indicator value is higher. Therefore, a change of a first motion pattern may be detected earlier.

According to a further embodiment, the method includes the following step performed by the control unit of the magnetic resonance device for each current first motion period.

The step includes a processing of the at least one the first motion component by a signal processing procedure to shift the phase of the respective first motion component to generate a second first motion component. In other words, at least one first motion component is phase shifted by a signal processing procedure. In other words, the at least one first motion component is processed by the control unit according to the signal processing procedure to change the phase of the respective first motion component. The signal processing procedure may include a derivation and/or an integration of the at least one first motion component. Therefore, the phase of the processed first motion component may advance or retard the original phase.

A second aspect is related to a method for correcting an amplitude of a motion trigger signal of a current second motion period of a cyclic second motion. The cyclic second motion may be a cardiac motion.

The method includes the steps of the method of the first aspect. In other words, during the method for correcting the amplitude of the trigger signal, the method for determining a first motion pattern of a subsequent first motion period of a current first motion period of a cyclic first motion of an object is performed.

For each current second motion period, a predicted amplitude of the trigger signal of the current second motion period is determined by the control unit. The evaluation of the predicted amplitude depends on the first motion pattern that is identified for the subsequent first motion period that is simultaneous to the current second motion period. In other words, to allow a correction of the amplitude of the trigger signal of the subsequent second motion period in Real-time, it is necessary to use the predicted amplitude of the subsequent second motion period, as the amplitude has to be known in advance. However, the predicted amplitude of the trigger signal of the subsequent second motion period may depend on the first motion pattern of the subsequent first motion period parallel to the subsequent second motion period. Therefore, the first motion pattern of the subsequent first motion period is acknowledged in the determination of the predicted amplitude.

For each current second motion period a second motion component of the pilot tone raw signal is extracted by the control unit of the magnetic resonance device. In other words, the component of the pilot tone raw signal that is related to the second cyclic motion is determined.

In a next step, the trigger signal is evaluated as a function of the second motion component of the pilot tone raw signal. The trigger signal describes the first inverse derivative of the second motion component of the pilot tone raw signal. In other words, the first inverse derivative of the second motion component is evaluated by the control unit. The first inverse derivative is used as the trigger signal.

In a next step, the control unit corrects the amplitude of the trigger signal of the current second motion period as a function of the predicted amplitude of the trigger signal of the current second motion period. In other words, the amplitude of the trigger signal is corrected in real time. It may be possible that the amplitude of the trigger signal of the current second motion period is normalised. As a reference for the normalisation the predicted amplitude of the trigger signal of the current second motion period is used.

In addition, or as an alternative, the threshold amplitude value of the current second motion period is corrected as a function of the predicted amplitude of the trigger signal (PTC) of the current second motion period.

The embodiment has the advantage that the triggering is corrected as a function of the subsequent first motion pattern. Therefore, delayed or missed triggers may be reduced.

According to a further embodiment, the method includes an evaluation of the predicted amplitude of the trigger signal of the current second motion period as a function of a detected amplitude of the trigger signal of selected second motion periods. In other words, the predicted amplitude of the trigger signal of the current second motion period is based on detected amplitudes of the trigger signal of preceding selected second motion periods.

According to a further embodiment, the method includes a detection of the trigger. The trigger is detected if the threshold amplitude value is exceeded by the corrected amplitude of the trigger signal. In other words, the trigger relates to the exceeding of the threshold amplitude by the corrected amplitude of the trigger signal.

A third aspect is related to a magnetic resonance device including a control unit.

The magnetic resonance device is configured to perform a method according to the first aspect.

The control unit is configured to receive a raw data signal acquired by a magnetic resonance receiver coil assembly of the magnetic resonance device and to extract a pilot tone raw signal from the raw data signal. The control unit is configured to extract at least one first motion component from the pilot tone raw signal and to determine a respective first motion signal based on the at least one first motion component. The first motion signal may be a first derivative of the at least one first motion component, or alternatively, the cleaned-up component itself. The control unit is configured to determine a determined activity indicator value of a current first motion period. The determined activity indicator value may depend on a slope of the respective first motion signal. The determined activity indicator value may be a maximum amplitude value of the respective first motion signal of the current first motion period. The control unit is configured to determine a predicted activity indicator value of a subsequent first motion period based on the determined activity indicator value of the current first motion period; and to identify the first motion pattern of the subsequent first motion period as a function of the predicted activity indicator value of the subsequent first motion period.

According to a further embodiment, the control unit is configured to evaluate a predicted amplitude of a trigger signal of a current second motion period depending on the first motion pattern of the subsequent first motion period that is simultaneous to the current second motion period. The control unit is configured to extract a second motion component of the pilot tone raw signal. The control unit is configured to evaluate the trigger signal that is a first inverse derivative of the second motion component of the pilot tone raw signal; and to correct an amplitude of the trigger signal of the current second motion period as a function of the predicted amplitude of the trigger signal of the current second motion period.

In addition, or as an alternative, the control unit is configured to correct the threshold amplitude value of the current second motion period as a function of the predicted amplitude of the trigger signal (PTC) of the current second motion period.

In other words, the magnetic resonance device is configured to perform a method according to the second aspect.

A fourth aspect is related to a computer program product including program code for performing a method according to the first aspect. The computer program product may also be regarded to a computer program.

A fifth aspect is related to a computer-readable storage medium including at least the computer program product according to the fourth aspect.

For use cases or use situations that may arise in the methods, and that are not explicitly described here, it may be provided that, in accordance with the methods, an error message and/or a prompt for user feedback is output and/or a default setting and/or a predetermined initial state is set.

Independent of the grammatical term usage, individuals with male, female or other gender identities are included within the term.

The control unit may include computing devices to perform steps of the methods.

A computing device may in particular be understood as a data processing device, that includes processing circuitry. The computing device may therefore in particular process data to perform computing operations. This may also include operations to perform indexed accesses to a data structure, for example a look-up table, LUT.

In particular, the computing device may include one or more computers, one or more microcontrollers, and/or one or more integrated circuits, for example, one or more application-specific integrated circuits, ASIC, one or more field-programmable gate arrays, FPGA, and/or one or more systems on a chip, SoC. The computing device may also include one or more processors, for example one or more microprocessors, one or more central processing devices, CPU, one or more graphics processing devices, GPU, and/or one or more signal processors, in particular one or more digital signal processors, DSP. The computing device may also include a physical or a virtual cluster of computers or other of said devices.

In various embodiments, the computing device includes one or more hardware and/or software interfaces and/or one or more memory devices.

A memory device may be implemented as a volatile data memory, for example a dynamic random access memory, DRAM, or a static random access memory, SRAM, or as a non-volatile data memory, for example a read-only memory, ROM, a programmable read-only memory, PROM, an erasable read-only memory, EPROM, an electrically erasable read-only memory, EEPROM, a flash memory or flash EEPROM, a ferroelectric random access memory, FRAM, a magneto resistive random access memory, MRAM, or a phase-change random access memory, PCRAM.

In the following, embodiments are explained in detail with reference to specific implementations and respective schematic drawings. In the drawings, identical or functionally identical elements may be denoted by the same reference signs. The description of identical or functionally identical elements is not necessarily repeated with respect to different figures.

### BRIEF DESCRIPTION OF THE FIGURES

FIG. 1 depicts a schematic illustration of a magnetic resonance device according to an embodiment.

FIG. 2 depicts a schematic illustration of a progression of the first motion signal according to an embodiment.

FIG. 3 depicts a schematic illustration of a progression of a trigger signal according to an embodiment.

FIG. 4 depicts a schematic illustration of a progression of a trigger signal of a corrected amplitude according to an embodiment.

FIG. 5 depicts a schematic illustration of a method for correcting an amplitude of a motion trigger signal according to an embodiment.

### DETAILED DESCRIPTION

The magnetic resonance device 1 includes a housing defining a bore and a main magnet arrangement, that is configured to generate a main magnetic field, also denoted as polarizing magnetic field, within the bore. The magnetic resonance device 1 includes an RF system, that is configured to apply an asymmetric RF pulse to a target material, in particular a body part of a patient, disposed within the bore and to receive MR signals from the target material. For example, the main magnet arrangement may generate a uniform main magnetic field B0 as the main magnetic field and at least one RF coil of the RF system may emit an excitation field B1. The magnetic resonance device 1 includes a data processing apparatus with at least one computing unit that is configured to construct the asymmetric RF pulse by using a computer implemented method for constructing an asymmetric RF pulse according to the present disclosure.

According to MR techniques, the target material is subjected to the main magnetic field, causing the nuclear spins in the target material to precess about the main magnetic field at their characteristic Larmor frequency. A net magnetic moment Mz is produced in the direction z of the main magnetic field, and the randomly oriented magnetic moments of the nuclear spins cancel out one another in the x-y-plane.

When the target material is then subjected to the transmit RF magnetic field, that is for example in the x-y plane and near the Larmor frequency, the net magnetic moment rotates out of the z-direction generating a net in-plane magnetic moment, that rotates in the x-y plane with the Larmor frequency. In response, MR signals are emitted by the excited spins when they return to their state before the excitation. The emitted MR signals are detected, for example by the at least one RF coil and/or one or more dedicated detection coils, digitized in a receiver channel of an RF controller 12 of the RF system, and processed by at least one processor of the at least one computing unit to reconstruct an MR image using a known MR reconstruction technique.

In particular, gradient coils 10 of the magnetic resonance device 1 may produce magnetic field gradients Gx, Gy, and Gz for position-encoding of the MR signals. Accordingly, MR signals are emitted only by such nuclei of the target material, that correspond to the particular Larmor frequency. For example, Gz is used together with a bandwidth-limited RF pulse to select a slice perpendicular to the z-direction and consequently may also be denoted as slice selection gradient. In alternative example, Gx, Gy, and Gz may be used in any predefined combination with a bandwidth-limited RF pulse to select a slice perpendicular to the vector sum of said gradient combination. The gradient coils 10 may be supplied with current by respective amplifiers for generating the respective gradient fields in x-direction, y-direction, and z-direction, respectively. Each amplifier may include a respective digital-to-analog converter, that is controlled by the sequence controller to generate respective gradient pulses at predefined time instances.

A sequence controller of the at least one computing unit may control the generation of RF pulses by an emitter channel of the RF controller and an RF power amplifier of the RF system.

The least one processor may receive the real and imaginary parts from analog-digital converters of the receiver channel and reconstruct the MR image based on them according to a known technique.

It is noted that each component of the magnetic resonance device 1 may include other elements that are required for the operation thereof, and/or additional elements for providing functions other than those described in the present disclosure.

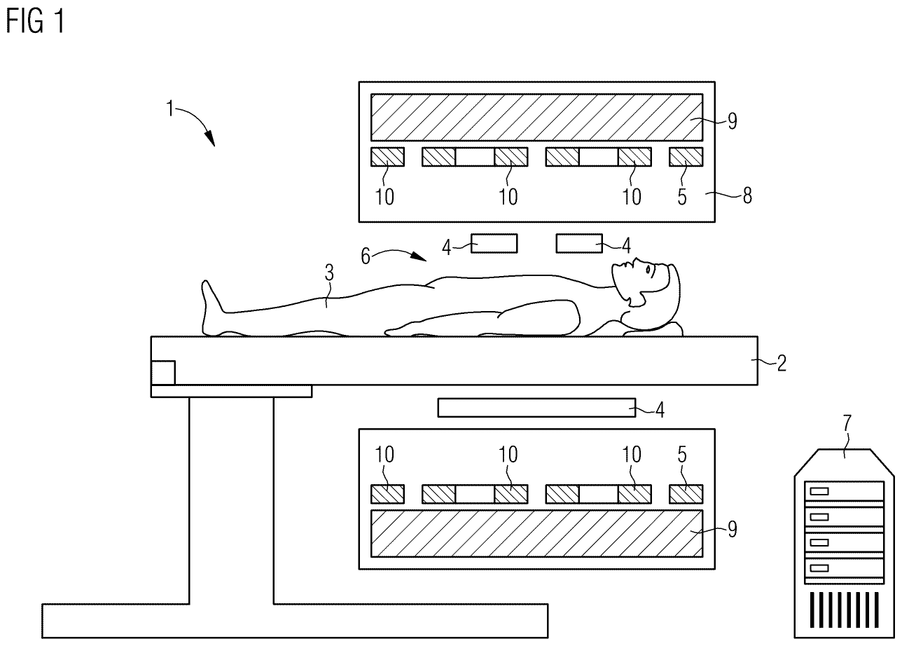

Furthermore, the magnetic resonance device 1 includes a pilot tone generator, that may be positioned close to a heart of the patient, for example close to the positions of the receiver coils 4 in FIG. 1. It is, however, also possible to locate the pilot tone generator in the patient table 2 or near the gradient coils 10.

The pilot tone generator 5 may be controlled to emit a pilot tone raw signal, in particular denoted as pilot tone, into the target region 6. The pilot tone raw signal is, in particular, off-band with respect to the received image acquisition signals that may have much higher bandwidth and band shifts due to the gradients active during reception. Therefore it is possible to extract the pilot tone raw signal from a raw data signal.

A magnetic resonance receiver coil 4 assembly of the magnetic resonance device 1 may be configured to receive the raw data signal. The raw data signal may include the image acquisition signal and the pilot tone raw signal. The magnetic resonance device 1 may be configured to provide the raw data signal to the control unit 7 of the magnetic resonance device 1. The control unit 7 may be configured to extract the pilot tone raw signal from the raw data signal. The extraction may be performed according to a predefined pilot tone extraction method known from the state of the art.

The pilot tone raw signal may include separate components that may be related to specific motions of the object 3. The motions may include cyclic motions. A first cyclic motion may be related to a respiratory motion of the object 3. A second cyclic motion may be related to a cardiac motion of the option. The second motion component may be used to trigger an acquisition of an image of the object 3 at a specific time point. Therefore, the control unit 7 may be configured to extract the second motion component from the pilot tone raw signal according to a predefined second motion component extraction method. The control unit 7 may be configured to evaluate a trigger signal PTC that may be a first inverse derivative of the second motion component of the pilot tone raw signal. In other words, the control unit 7 it may determine the first inverse derivative of the second motion component of the pilot tone raw signal to provide the trigger signal PTC. The trigger signal PTC may be used to trigger the acquisition of the image. The trigger signal PTC may be influenced by the current first motion pattern.

In particular an amplitude of the trigger signal PTC may depend on a current first motion pattern of this of the first cyclic motion. During specific first motion patterns of the first cyclic motion the amplitude of the trigger signal PTC may be reduced. The acquisition may be triggered when the amplitude of the trigger signal PTC is above a predefined threshold.

In order to avoid a missing of triggers, the control unit 7 may be configured to evaluate a predicted amplitude of the trigger signal PTC of the subsequent second motion period according to a trigger signal PTC amplitude prediction method. In other words, the control unit 7 may be configured to estimate the amplitude of the trigger signal PTC during the cure and second motion period. The second motion period may be related to a cardiac cycle. The trigger signal PTC amplitude prediction method may depend on the subsequent first motion pattern of the subsequent first motion period. In other words the trigger signal PTC amplitude prediction method used to predict the trigger signal PTC amplitude of the subsequent second motion period depends on the subsequent first motion pattern of the subsequent first motion period.

The control unit 7 is configured to correct the amplitude of the trigger signal PTC of the subsequent second motion period according to a predefined correction method as a function of the predicted amplitude of the trigger signal PTC of the subsequent second motion period. In other words the predicted amplitude is used by the control unit 7 to perform a correction of the amplitude of the trigger signal PTC during the subsequent second motion period.

The control unit 7 may observe the trigger signal PTC of securing second motion period and trigger an acquisition of image when the amplitude of the trigger signal PTC is above a predefined threshold value.

To determine the subsequent first motion pattern of the subsequent first motion period the control unit 7 is configured to extract a first motion component from the pilot tone raw signal in accordance with a predefined first motion component extraction method. In other words the control unit 7 is configured to identify a component of the pilot tone raw signal related to the first cyclic motion and to extract the first cyclic motion component from the pilot tone raw signal. The first cyclic motion component may be a dominant motion component of the pilot tone raw signal related to the first motion.

The control unit 7 is configured to evaluate the first motion signal PTR, that is a first derivative of the first motion component of the pilot tone raw signal. In other words, the control unit 7 derives the first motion component of the pilot tone raw signal to generate a first motion signal PTR.

The control unit 7 is configured to determine an indicator value of a first motion activity of the current first motion period, that is annexing value of an amplitude of the first motion signal PTR of the current first motion period. In other words, the control unit 7 is configured to analyze the amplitude of the first motion signal PTR that is recorded during the current first motion period. The control unit 7 may identify the maximum value of the amplitude that is reached by the first motion signal PTR during the current first motion period.

The control unit 7 may be configured to use the indicator value of the first motion activity of the current first motion period to determine men a predicted indicator value of the first motion activity of the subsequent and first motion period. The prediction indicator value may be evaluated according to a predefined indicator value prediction method. In other words based on the maximum value of the amplitude of the first motion signal PTR of the current first motion period a maximum amplitude value of the first motion signal PTR of the first motion period may be determined. In a next step the control unit 7 may be configured to identify the subsequent first motion pattern of the subsequent first motion period as a function of the predicted indicator value of the first motion activity of securing first motion period according to a predefined first motion pattern identification method. In other words the subsequent first motion pattern of the first motion during the subsequent first motion period may be determined based on the predicted indicator value that was predicted for the subsequent and first motion period.

The subsequent first motion pattern of securing first motion period may be identified by comparing the predicted indicator value of the first motion activity of securing first motion period to a predefined threshold value of the subsequent first motion period. The threshold value of securing first motion period may be based on indicator values of the first motion activity of selected previous first motion periods according to a predefined threshold determination method.

The selected previous first motion periods from previous first motion periods may be selected depending on the first motion pattern of the first motion periods. In other words the control unit 7 may identify the first motion patterns of the respective motion periods. The control unit 7 may be configured to store the indicator values that where determined for the respective first motion periods. Some of the indicator values may be suitable 2 for a generation of the threshold value. That may depend on the first motion pattern of the respective first motion period. The control unit 7 may be configured to identify the periods of the respective motion patterns and use the indicator values of the respective first motion patterns to generate the predefined threshold value.

The subsequent first motion pattern of the subsequent pattern may depend on the relation between the threshold and the predicted indicator value.

The control unit 7 may be configured to determine a length of the subsequent first motion period and to identify the subsequent first motion pattern of the subsequent first motion as a function of the length of the current first motion period. The subsequent first motion pattern of the subsequent first motion period may be identified by the control unit 7 as a function of the first motion pattern of the current first motion period.

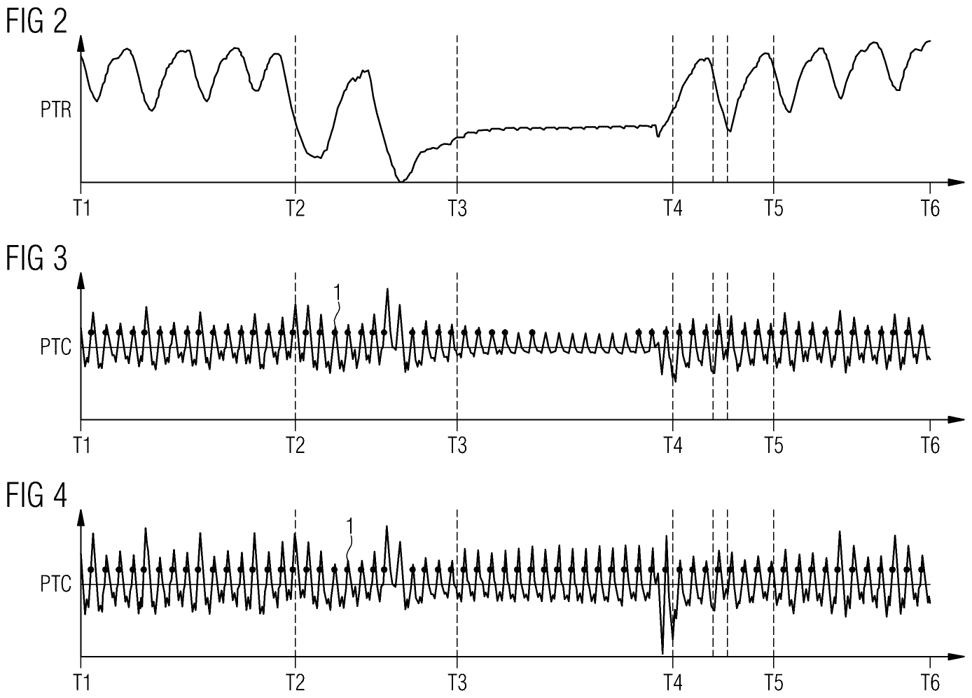

FIG. 2 depicts a schematic illustration of a progression of the first motion signal PTR.

The first motion signal PTR may be used to determine the first motion pattern of the subsequent first motion period, following the current first motion period. As the first motion may be related to breathing of the object 3, the first motion patterns may be related to respective breathing patterns or breathing patterns. A first range from T1 to T2 may include first motion periods that may be identified as free briefing first motion patterns. A second range from T2 to T3 may include first motion periods that may be identified as deep breathing first motion patterns. A third range from T3 to T4 may include first motion periods that may be identified as breath hold first motion patterns. A fourth range from T4 to T5 may include first motion periods that may be identified as deep breathing first motion patterns again. A fifth range from T5 to T6 may include first motion periods that may be identified as free breathing first motion patterns again.

FIG. 3 depicts a schematic illustration of a progression of a trigger signal PTC.

The second row depicts a progression of the trigger signal PTC of the second motion. The illustration depicts the ranges, already shown in FIG. 3 to demonstrate the influence of the first motion on the second motion. The second motion may be related to a cardiac motion. The trigger signal PTC may be used to trigger an acquisition of the image of the object 3. The acquisition may be triggered at respective trigger points 11. The trigger points 11 may be times, when the amplitude of the trigger signal PTC exceeds a predefined trigger amplitude threshold. It may be intended that each of the second motion periods includes a trigger point 11. FIG. 3 depicts that during the third range from T3 to T4 including first motion periods that may be identified as breath hold first motion patterns, the amplitude of the trigger signal PTC is relatively low, compared to the other ranges. Therefore the amplitude may not reach the amplitude threshold during some of the second motion periods. The respective second motion periods may not include trigger points 11. FIG. 3 depicts the influence of the first motion on the second motion. Therefore the amplitude may be corrected to compensate the lower amplitude during the breath hold first motion patterns.

FIG. 4 depicts a schematic illustration of a progression of a trigger signal PTC of a corrected amplitude.

FIG. 4 depicts the trigger signal PTC of FIG. 3 with the correct amplitude. The correction of the amplitude depends on the first motion pattern during the respective second motion period. It may be seen that the correction of the amplitude causes an increase of the amplitude in the third range from T3 to T4 related to a breath hold. Therefore every second motion period during the third range includes a trigger point 11.

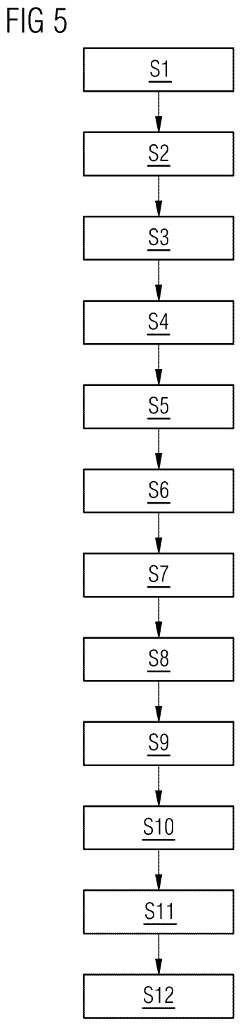

FIG. 5 depicts a schematic illustration of a method for correcting an amplitude of a motion trigger signal PTC.

The first motion may be related to a breathing of the object 3, wherein the second motion may be related to a cardiac motion of the object 3. The aim of the method may be a consideration of an influence of the breathing on the cardiac motion. The first motion and the second motion may occur in parallel. The method may be performed by the magnetic resonance device 1 shown in FIG. 1.

The following steps S1 to S7 may be steps of a method for determining a first motion pattern of a subsequent first motion period of a current first motion period of a cyclic first motion of an object 3 by a magnetic resonance device 1.

For each current first motion period, the control unit 7 of a magnetic resonance device 1 may receive a raw data signal acquired by the magnetic resonance receiver coil 4 assembly of the magnetic resonance device 1 in a step S1.

In a step S2, the control unit 7 may extract a pilot tone raw signal of the current first motion period from the raw data signal.

In a step S3, the control unit 7 may extract at least one first motion component from the pilot tone raw signal of the current first motion period.

In a step S4, the control unit 7 may determine a respective first motion signal PTR of the current first motion period that is a first derivative of the at least one first motion component.

In a step S5, the control unit 7 may determine a respective determined activity indicator value of the current first motion period, that is a maximum amplitude value of the respective first motion signal PTR of the current first motion period.

In a step S6, the control unit 7 may determine a respective predicted activity indicator value of the subsequent first motion period based on the determined activity indicator value of the current first motion period.

In a step S7, the control unit 7 may identify the first motion pattern of the subsequent first motion period as a function of the predicted activity indicator value of the subsequent first motion period.

The following steps S8 to S12 may be steps of a method for correcting an amplitude of a motion trigger signal PTC of a current second motion period of a cyclic second motion.

For each current second motion period, the control unit 7 of the magnetic resonance device 1 may evaluate a predicted amplitude of a trigger signal PTC of the current second motion period depending on the first motion pattern of the subsequent first motion period that is simultaneous to the current second motion period in a step S8. The prediction of the predicted amplitude of the trigger signal PTC of the current second motion period may have a stiffness depending on the predicted first motion pattern. In other words, an amount of correction may depend on the predicted first motion pattern. In some of the first motion patterns there may be no correction of the amplitude.

In a step S9, the control unit 7 may extract a pilot tone raw signal of the current second motion period.

In a step S10, the control unit 7 may extract a second motion component of the pilot tone raw signal of the current second motion period.

In a step S11, the control unit 7 may evaluate the trigger signal PTC of the current second motion period that is a first inverse derivative of the second motion component of the pilot tone raw signal.

In a step S11, the control unit 7 may correct an amplitude of the trigger signal PTC of the current second motion period as a function of the predicted amplitude of the trigger signal PTC of the current second motion period.

In a step S12, the control unit 7 may detect an exceeding of the predefined amplitude threshold by the corrected amplitude of the trigger signal PTC. The control unit 7 may trigger an acquisition of an image by the magnetic resonance device 1 of may trigger a generation of an image based on image signals, recorded at a time of triggering.

The approach is to determine in real-time the current corrected signal amplitude of the trigger signal PTC based solely on pilot tone data. The trigger signal PTC, also known as Beat Sensor signal, may be the inverted derivative of the second motion component, also known as cardiac component, in the pilot tone raw signal. The trigger signal PTC may be generated by weighted linear combination of the raw pilot tone signal followed by calculation of a stable 2 derivative in a constant velocity Kalman filter.

The determined trigger signal PTC amplitude that may be determined in one second motion period, giving a cardiac period related to one heartbeat may only be applied as the predicted trigger signal PTC amplitude to the following cardiac period. A correction will fail if respective determined trigger signal PTC amplitudes of successive cardiac periods are uncorrelated, especially, if a change in determined trigger signal PTC amplitudes is not smooth. During patterns of the rapidly changing amplitudes of the trigger signal PTC the of the predicted trigger signal PTC amplitude must be stabilized, e.g, by averaging over several previous second motion periods, by a pass filter, by reducing contributions of outliners, or the update of the predicted trigger signal PTC amplitude must be suspended completely.

Because the trigger signal PTC amplitude variation is caused by respiratory motion, the stabilization of the predicted trigger signal PTC amplitude estimate may be adjusted based on the first motion component from the pilot tone raw signal. Only a strongest first motion component from the pilot tone raw signal may be used and a stable second derivative of the first motion component from the pilot tone raw signal being a first motion signal PTR may be calculated using a second constant velocity Kalman filter that may be independent of slow signal drifts in the different pilot tone receive channels. A magnitude of maxima between zero crossings of the first motion signal PTR may be used as a determined activity indicator value of respiratory activity that may be updated every respiratory half cycle. From this determined activity indicator value a slowly varying threshold value may be evaluated by low pass filtering and suspension of update during specific first motion patterns. First motion pattern of a subsequent first motion period may be identified by comparing the predicted activity indicator value to the threshold value of the subsequent first motion period. First motion pattern of the subsequent first motion period may also be identified by a comparison of the predicted activity indicator value with an average respiratory activity during training. The determined activity indicator value may be determined at discrete times. It may be calculated every respiratory half cycle. The span of a respiratory half-cycle may be determined either by zero crossing of the first motion signal PTR or by a value established during training, whichever comes first. Thus, the determined activity indicator value of the current respiratory half cycle may be the predicted activity indicator value of the subsequent first motion period. The evaluation of the predicted activity indicator value estimate may be improved by model-based interpolation of the discrete determined activity indicator values.

The first motion patterns may include three predefined first motion patterns that may relate to three different respiratory patterns.

One of the first motion patterns may be related to a free breathing. This first motion pattern may relate to a natural breathing pattern of the patient at rest. The training procedure may take place during this first motion pattern. During this first motion pattern the trigger signal PTC amplitude varies only slightly but rather quickly with the first motion period, that is typically 4-10 s long.

Therefore the predicted activity indicator value may be calculated using a low-pass filter that smoothens the variations inside a first motion pattern sufficiently.

One of the first motion patterns may be related to a breath hold. This first motion pattern may be distinguished between inspiration and expiration utilizing characteristics of the first motion signal PTR, e.g., the direction that respiratory velocity approaches the value during breath hold. During this time, the respiratory activity is very low but drifts in determined activity indicator value and/or trigger signal PTC may occur, e.g., due to bad breath hold compliance or internal organ motion, so called diaphragm deformation). Thus, trigger signal PTC amplitude may vary slowly, and predicted amplitude of a trigger signal PTC of the current second motion period based on the current amplitude of the trigger signal PTC will be precise.

One of the first motion patterns may be related to a deep breathing. This first motion pattern occurs typically before and after a breath hold first motion pattern to prepare for and recover from the breath hold first motion pattern. During that period, no imaging is performed and the goal is not to detect precise triggers, but to avoid that the predicted amplitude of the trigger signal PTC is worsened for the first motion pattern related to breath hold or, less important, free breath. Therefore, the update of the predicted amplitude of the trigger signal PTC is suspended during this first motion pattern.

There may be several approaches to identify the first motion pattern.

The identification of the first motion patterns may be based on training data recorded during a free breath pattern of the object 3 in a training procedure. During training procedure individual-specific features may be extracted that characterize respiratory activity during the free breath the first motion pattern, such as respiratory half-cycle length and detected activity indicator value standard deviation as measure for average detected activity indicator value during free breath are extracted. The direction of the first motion signal PTR may also be established during this pattern.

A start of a breath hold first motion pattern may be detected by comparing the predicted activity indicator value estimated for the subsequent first motion period with a cutoff value. If the current first motion period, related to the preceding respiratory half-cycle is identified as a deep breath or a breath hold first motion pattern, the cutoff amplitude may be 0.4 times the threshold value, otherwise the cutoff value may be 0.2 times the threshold.

An end of a breath hold first motion pattern may be detected, when the predicted activity indicator value estimated for the subsequent first motion period exceeds the cutoff value of 0.4 times the threshold value.

A start of a deep breath first motion pattern may be detected, when the predicted activity indicator value estimated for the subsequent first motion period is greater than the threshold by five standard deviations, that is established during training.

An end of a deep breath first motion pattern may be detected, when the predicted activity indicator value estimated for the subsequent first motion period falls below the currently described cutoff value.

A determination of the current cut off value may depend on the current first motion pattern. During a free breath, the current cut off value may be determined by using a low pass filter, e.g., exponential smoothing with factor 0.3.

During a breath hold, the current cut off value may be kept constant.

During a deep breath, the current cut off value may be kept constant or may vary slowly.

The analysis of the first cyclic motion may be performed based on the dominant first motion component. However a restriction to the dominant first motion component may limit updates for activity indicator values to half cycles of the dominant first motion component. This update rate may be increased by adding information from further first motion components that may be pattern shifted to the dominant first motion component. If the pattern shift between the further first motion component and the dominant first motion component is 90 degrees, the update rate could be approximately doubled compared to an analysis based on the dominant first motion component alone. The further first motion component may be the respiratory signal itself, but it may be used only if signal drift has been compensated or is small and slow enough not to disturb the determination of the determined activity indicator value. The further first motion components and their derivatives could also be used as signals pattern shifted to the first motion component because it has been shown that these components may correspond to geometrical changes that may occur during the respiratory motion for example relate to the respiratory hysteresis.

It is to be understood that the elements and features recited in the appended claims may be combined in different ways to produce new claims that likewise fall within the scope of the present disclosure. Thus, whereas the dependent claims appended below depend from only a single independent or dependent claim, it is to be understood that the dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for determining a first motion pattern of a subsequent first motion period of a current first motion period of a cyclic first motion of an object by a magnetic resonance device, the method comprising, for each current first motion period:
receiving, by a control unit of a magnetic resonance device, a raw data signal acquired by a magnetic resonance receiver coil assembly of the magnetic resonance device;
extracting, by the control unit, a pilot tone raw signal from the raw data signal;
extracting, by the control unit, at least one first motion component from the pilot tone raw signal;
determining, by the control unit, a respective first motion signal based on the at least one first motion component;
determining, by the control unit, a determined activity indicator value of the current first motion period, depending on a slope of the respective first motion signal of the current first motion period;
determining, by the control unit, a predicted activity indicator value of the subsequent first motion period based on the determined activity indicator value of the current first motion period; and
identifying, by the control unit, the first motion pattern of the subsequent first motion period as a function of the predicted activity indicator value of the subsequent first motion period.

2. The method of claim 1, wherein the respective first motion signal is a first derivative of the at least one first motion component; and wherein the determined activity indicator value of the current first motion period is a maximum amplitude value of the respective first motion signal of the current first motion period.

3. The method of claim 1, wherein the respective first motion signal is a cleaned-up version of the at least one first motion component; and wherein the determined activity indicator value of the current first motion period is a mean or maximum amplitude value of the respective first motion signal of the current first motion period.

4. The method of claim 1, further comprising a training procedure, the training procedure comprising:
recording, by the control unit, the first motion signal during a training period associated with a predefined first motion pattern; and
evaluating, by the control unit, at least one identification characteristic for identification of the predefined first motion pattern based on the first motion signal recorded during the training period, the at least one identification characteristic comprising a length of a first motion period of the predefined first motion pattern and/or an average activity indicator value of the predefined first motion pattern and/or a standard deviation of the activity indicator value of the predefined first motion pattern and/or a direction of the respective first motion signal of the predefined first motion pattern.

5. The method of claim 4, further comprising for each current first motion period:
identifying, by the control unit, the first motion pattern of the subsequent first motion period as a function of the at least one identification characteristic.

6. The method of claim 1, further comprising for each current first motion period:
comparing, by the control unit, the predicted activity indicator value of the subsequent first motion period to a predefined threshold value of the subsequent first motion period; and
identifying, by the control unit, the first motion pattern of the subsequent first motion period depending on a result of the comparison.

7. The method of claim 6, further comprising for each current first motion period:
determining, by the control unit, the threshold value of the subsequent first motion period based on determined activity indicator values of selected first motion periods.

8. The method of claim 7, further comprising for each current first motion period:
selecting, by the control unit, the selected first motion periods from the first motion periods depending on the first motion pattern of the first motion periods.

9. The method of claim 1, further comprising for each current first motion period:
identifying, by the control unit, the first motion pattern of the subsequent first motion period as a function of a length of the current first motion period.

10. The method of claim 1, further comprising for each current first motion period:
identifying, by the control unit, the first motion pattern of the subsequent first motion period as a function of the first motion pattern of the current first motion period.

11. The method of claim 1, further comprising for each current first motion period:
extracting, by the control unit, at least two of the first motion components from the pilot tone raw signal.

12. The method of claim 1, wherein the at least two of the first motion components are phase-shifted to each other.

13. The method of claim 1, further comprising for each current first motion period:
processing, by the control unit, the at least one first motion component by a signal processing procedure to shift a phase of the respective first motion component to generate a phase shifted second first motion component.

14. The method of claim 1, further comprising:
correcting, by the control unit, a triggering by a motion trigger signal of a current second motion period of a cyclic second motion, wherein correcting comprises for each current second motion period:
evaluating a predicted amplitude of a trigger signal of the current second motion period, depending on the first motion pattern of the subsequent first motion period that is simultaneous to the current second motion period;
extracting a second motion component of the pilot tone raw signal;
evaluating the trigger signal that is a first inverse derivative of the second motion component of the pilot tone raw signal; and
correcting an amplitude of the trigger signal of the current second motion period as a function of the predicted amplitude of the trigger signal of the current second motion period and/or correcting a threshold amplitude value of the current second motion period as a function of the predicted amplitude of the trigger signal of the current second motion period.

15. The method of claim 14, further comprising for each current second motion period:
evaluating, by the control unit, the predicted amplitude of the trigger signal of the current second motion period, as a function of an amplitude of the trigger signal of selected second motion periods.

16. The method of claim 14, further comprising for each current second motion period:
detecting, by the control unit, a trigger if the threshold amplitude value is exceeded by the amplitude of the trigger signal.

17. A magnetic resonance device, comprising:
a magnetic resonance receiver coil assembly; and
a control unit wherein the control unit is configured to:
receive a raw data signal acquired by the magnetic resonance receiver coil assembly;
extract a pilot tone raw signal from the raw data signal;
extract at least one first motion component from the pilot tone raw signal;
determine a respective first motion signal based on the at least one first motion component;
determine a determined activity indicator value of a current first motion period, depending on the respective first motion signal of the current first motion period;
determine a predicted activity indicator value of a subsequent first motion period based on the determined activity indicator value of the current first motion period; and
identify a first motion pattern of the subsequent first motion period as a function of the predicted activity indicator value of the subsequent first motion period.

18. The magnetic resonance device of claim 17, wherein the control unit is further configured to:
evaluate a predicted amplitude of a trigger signal of a current second motion period, depending on the first motion pattern of the subsequent first motion period that is simultaneous to the current second motion period;
extract a second motion component of the pilot tone raw signal;
evaluate the trigger signal that is a first inverse derivative of the second motion component of the pilot tone raw signal; and
correct an amplitude of the trigger signal of the current second motion period as a function of the predicted amplitude of the trigger signal of the current second motion period and/or to correct a threshold amplitude value of the current second motion period as a function of the predicted amplitude of the trigger signal of the current second motion period.

19. A non-transitory computer implemented storage medium that stores machine-readable instructions executable by at least one processor, the machine-readable instructions comprising:
receiving a raw data signal acquired by a magnetic resonance receiver coil assembly of a magnetic resonance device;
extracting a pilot tone raw signal from the raw data signal;
extracting at least one first motion component from the pilot tone raw signal;
determining a respective first motion signal based on the at least one first motion component;
determining a determined activity indicator value of a current first motion period, depending on the respective first motion signal of the current first motion period;
determining a predicted activity indicator value of a subsequent first motion period based on the determined activity indicator value of the current first motion period; and
identifying a first motion pattern of the subsequent first motion period as a function of the predicted activity indicator value of the subsequent first motion period.

20. The non-transitory computer implemented storage medium of claim 19, wherein the machine-readable instructions further comprising:
evaluating a predicted amplitude of a trigger signal of a current second motion period, depending on the first motion pattern of the subsequent first motion period that is simultaneous to the current second motion period;
extracting a second motion component of the pilot tone raw signal;
evaluating the trigger signal that is a first inverse derivative of the second motion component of the pilot tone raw signal; and
correcting an amplitude of the trigger signal of the current second motion period as a function of the predicted amplitude of the trigger signal of the current second motion period and/or to correct a threshold amplitude value of the current second motion period as a function of the predicted amplitude of the trigger signal of the current second motion period.
